(19) United States
(12) Patent Application Publication
(10) Pub. No.: US 2010/0274103 A1
(43) Pub. Date: Oct. 28, 2010

(54) ULTRASOUND COMMUNICATIONS VIA WIRELESS INTERFACE TO PATIENT MONITOR

(75) Inventors: Allen David Snook, Snohomish, WA (US); Bryce Loren Greenhalgh, Woodinville, WA (US); Dan M. Skyba, Snohomish, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/681,178

(22) PCT Filed: Oct. 6, 2008
(86) PCT No.: PCT/IB08/54090

§ 371 Date: Apr. 1, 2010

Related U.S. Application Data
(60) Provisional application No. 60/978,786, filed on Oct. 10, 2007.

(51) Int. Cl. A61B 5/02

(52) U.S. Cl. 600/301

(57) ABSTRACT

An ultrasound diagnostic imaging system (10) is provided that includes a patient monitor (12) and an ultrasound machine (14). The patient monitor (12) includes a communications processing unit (16) with a sensor interface (18) for receiving physiological data and a wireless interface (20) for transmitting ultrasound control data to the ultrasound machine (14). The patient monitor (12) further includes a data processing unit (24) that generates ultrasound control data in response to physiological data received by the sensor interface (18). The ultrasound machine (14) includes a communications processing unit (30) with a wireless interface (32). The communications processing units (16, 30) establish a low-latency wireless communication link that allows ultrasound control data sent by the patient monitor (12) to trigger the acquisition of ultrasound frames, images, and/or volumes at an appropriate time during a physiological cycle.

The present disclosure relates to systems and methods for ultrasonic diagnostic imaging. More particularly, the present disclosure is directed to ultrasonic apparatus/systems and related methods that include and/or facilitate use of an ultrasound machine in wireless communication with a patient monitor that receives physiological data and generates control data used to trigger acquisition of ultrasound frames, images and/or volumes at a particular time during a physiological cycle.

Ultrasonic diagnostic imaging systems allow medical professionals to examine internal structures of patients without invasive exploratory surgery. Ultrasonic diagnostic imaging systems typically include a variety of types of transducer probes, each having different ultrasound data acquisition capabilities, operating characteristics and/or modes of operation. A transducer probe is typically connected to an ultrasound machine that provides control signals to the transducer probe, processes data acquired by the transducer probe, and displays a corresponding image.

Ultrasonic diagnostic imaging systems used for cardiac imaging are typically attached to sensors that acquire physiological data used to trigger the acquisition of ultrasound frames, images and/or volumes at an appropriate time during a cardiac cycle. Such physiological data could include electrocardiogram, R-wave indicators, respiration, pulse and/or oximetry data.

In many cases, a patient has similar sensors attached that supply electrocardiogram, R-wave indicators, respirations, pulse and/or oximetry data to a patient monitor. However, conventional patient monitors do not communicate physiological data to conventional ultrasound machines. Thus, a second set of sensors must be applied to the patient to supply physiological data to the ultrasound machine for triggering frame, image and/or volume acquisition. This arrangement requires additional work for the physician or attending staff who must keep all of the sensors in place. The patient also is inconvenienced by having additional sensors attached to her body. Further, the overall system cost is increased as multiple sensors that gather the same type of physiological data are required.

Thus, a need exists for improved monitoring systems and techniques for obtaining and processing relevant information from patients. These and other needs are satisfied by the systems and methods of the present disclosure.

The present disclosure provides advantageous apparatus/systems and methods that include a patient monitor with a wireless interface/transceiver that communicates with a complementary wireless interface/transceiver associated with an ultrasound machine. The patient monitor is typically attached to or otherwise in communication with sensors that are adapted to acquire physiological data. The patient monitor provides control signals to the ultrasound machine based on the acquired physiological data and such signals may advantageously trigger responsive actions/activities, e.g., the acquisition of ultrasound frames, images and/or volumes at an optimum time for diagnostic imaging.

The disclosed apparatus/systems and methods provide numerous advantages and benefits. For example, the disclosed apparatus/systems and methods obviate the need to attach additional sensors to a patient for triggering an ultrasound machine. Additional features, functions and benefits of the disclosed apparatus/systems and methods will be apparent from the detailed description which follows, particularly when read in conjunction with the appended figures.

To assist those of skill in the art in making and using the disclosed apparatus/systems and related methods, reference is made to the accompanying FIGURE, wherein:

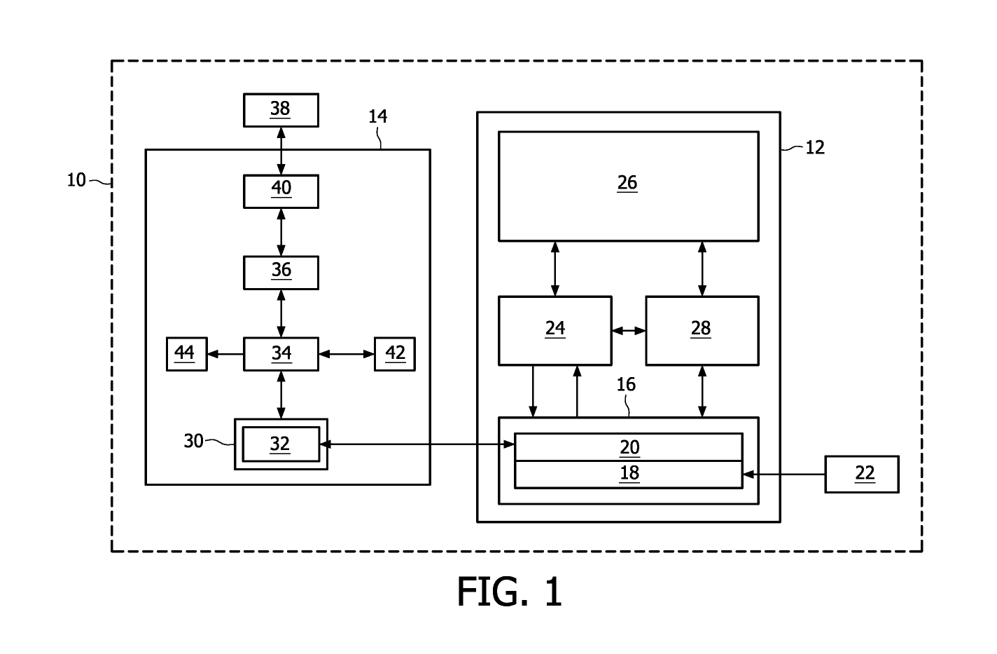

FIG. 1 is a schematic depiction of an exemplary ultrasound diagnostic imaging system made in accordance with the present disclosure.

In accordance with the exemplary embodiments of the present disclosure, an ultrasound diagnostic imaging system is provided for anatomical imaging that includes a patient monitor and an ultrasound machine. The patient monitor receives physiological data and generates control signals that are provided over a private wireless communication link to the ultrasound machine triggering the acquisition of desired information, e.g., ultrasound frames, images and/or volumes at an appropriate time during a cardiac cycle.

Referring now to FIG. 1, an exemplary ultrasound diagnostic imaging system is generally indicated at 10. The ultrasonic diagnostic system 10 includes a patient monitor 12 and an ultrasound machine 14.

The patient monitor 12 typically includes a communications processing unit 16 having a sensor interface 18 and a wireless interface 20. The communications processing unit 16 could be implemented using one or more general purpose processors, Application-Specific Integrated Circuits (ASICs), Field-Programmable Gate Arrays (FPGAs) and/or electronic storage devices known in the art.

A sensor 22 is generally adapted to provide physiological data to the sensor interface 18. The physiological data could include electrocardiogram, R-wave indicator, respiration, pulse and/or oximetry data, for example. In some embodiments, the sensor interface 18 includes a plurality of connectors for connecting a plurality of sensors 22 that provide more than one type of physiological data to the patient monitor 12. The wireless interface 20 implements a short range wireless communications protocol such as Bluetooth 2.1, Wi-Fi or IEEE 802.11, for example.

The communications processing unit 16 of the patient monitor 12 supplies physiological data received by the sensor interface 18 to a data processing unit 24. The communications processing unit 16 and the data processing unit 24 could be implemented using one or more general purpose processors, ASICs, FPGAs and/or electronic storage device, as are known in the art. The data processing unit 24 may be adapted to provide image data corresponding to the received physiological data to a display unit 26 for presentation to a medical professional (not shown). The medical professional could actuate a user interface 28 to control display characteristics of the display unit 26 or otherwise interact with the displayed results, e.g., by printing, storing or otherwise manipulating the image thereof.

The data processing unit 24 of the patient monitor 12 typically programmed to analyze/process the received physiological data. Based on such analysis/processing, data processing unit 24 may be programmed to determine and/or control operating parameters of ultrasound machine 14, e.g., timing, operating characteristics and/or modes of operation for ultrasound machine 14 to acquire ultrasound frames, images and/or volumes at an appropriate time during a cardiac cycle. The data processing unit 24 is also typically programmed to generate corresponding control data that is provided to the wireless interface 20 of the communications processing unit 16 for wireless transmission to the ultrasound machine 14. Such control data may be used to trigger acquisition of ultrasound frames, images and/or volumes at an appropriate time during a cardiac cycle, and/or to initiate/control other aspects of ultrasound operation.

The ultrasound machine 14 generally includes a communications processing unit 30 that includes a wireless interface 32. The wireless interface 32 implements a short range wireless communications protocol such as Bluetooth 2.1, Wi-Fi or IEEE 802.11, for example. The communications processing unit 16 of the patient monitor 12 and the communications processing unit 30 of the ultrasound machine 14 are generally adapted to exchange data using a known communications protocol, such as the Internet Protocol, Dynamic Host Configuration Protocol, Transmission Control Protocol, User Datagram Protocol, Wireless Encryption Protocol and/or Wi-Fi Protected Access.

The communications processing unit 30 is typically programmed to provide control data received by the wireless interface 32 to a controller unit 34. The controller unit 34 processes the control data and generates corresponding signals that are provided to a microbeamformer unit 36. Control signals transmitted to microbeamformer unit 36 may be effective to cause transducer probe 38 to acquire ultrasound frames, images and/or volumes at an appropriate time during a cardiac cycle, and/or to undertake other operational steps and/or activities.

In exemplary embodiments of the present disclosure, microbeamformer unit 36 performs microbeamforming operations on echo signals received from a transducer probe 38 through a transducer interface 40. The microbeamformer unit 36 may advantageously apply appropriate delays and may function to combine per-element echo signals into a small number of partially beamformed signals. For example, transducer probe 38 could include 128 individual transducer elements (not shown) and the microbeamformer unit 36 could receive echo signals from such individual transducer elements, apply appropriate delays, and combine the received echo signals to form eight partially beamformed signals. The communications processing unit 30, controller unit 34, and microbeamformer unit 36 could be implemented using one or more general purpose processors, ASICs, FPGAs and/or electronic storage device, as are known in the art.

The microbeamformer unit 36 generally provides beamformed echo signals to the controller unit 34, which transmits the beamformed echo signals through the wireless interface 32 to the patient monitor 12. The beamformed echo signals also could be stored, further processed and/or displayed by the ultrasound machine 14 (or associated components). The microbeamformer unit 36 also could be implemented to produce fully beamformed signals from all transducer elements of an active aperture, as described, for example, in U.S. Pat. No. 6,142,946 to Hwang et al. Microbeamformer technology suitable for use in microbeamformer unit 36 is described, for example, in U.S. Pat. No. 5,229,933 to Larson, III; U.S. Pat. No. 6,375,617 to Fraser; and U.S. Pat. No. 5,997,479 to Savord et al.

The ultrasound machine 14 may include a variety of types of transducer probes 38 that could be attached to transducer interface 40. The transducer probes 38 could include, for example, various types of ultrasonic image data acquisition transducer arrays (not shown), e.g., planar arrays, linear arrays and curved arrays, each of which could be operated as a phased array wherein relative phases of acoustic signals emitted by the transducer elements are varied to produce a desired radiation pattern.

The controller unit 34 and microbeamformer unit 36 typically generate control data appropriate for the particular type of transducer probe(s) 38 that is/are attached to the transducer interface 40. Similarly, the data processing unit 24 of patient monitor 12 is generally programmed to assess the particular type of transducer probe(s) 38 attached to the transducer interface 40 and generate control data appropriate for the particular type of transducer probe(s) 38 that is/are attached to transducer interface 40.

Moreover, each transducer probe 38 could have different operating characteristics that include center frequencies, elevation foci and fundamental versus harmonic performance, for example. Further, each transducer probe 38 could have one or more modes of operation, including fundamental imaging, harmonic imaging, B-mode imaging, Doppler imaging and/or color imaging, for example.

In exemplary embodiments of the present disclosure, the controller unit 34 and microbeamformer unit 36 are adapted to generate control data appropriate for the operating characteristics and mode(s) of operation of the particular transducer probe 38 that is attached to the transducer interface 40. In addition, the data processing unit 24 of the patient monitor 12 is typically programmed to assess the operating characteristics and mode(s) of operation of transducer probe 38 attached to the transducer interface 40 and generate appropriate control data that is transmitted to the ultrasound machine 14.

The data processing unit 24 may advantageously function to obtain the type, operating characteristics and/or modes operation of the transducer probe 38 attached to the transducer interface 40 in a variety of ways. For example, the type, operating characteristics and/or modes operation of the transducer probe 38 attached to the transducer interface 40 could be input or selected from a list (e.g., from a drop-down menu) using the user interface 28 and communicated to the data processing unit 24 of the patient monitor 12.

Alternatively, data indicating the type, operating characteristics and/or modes of operation of the transducer probe 38 attached to the transducer interface 40 could be transmitted from the ultrasound machine 14 to the patient monitor, for example, during a communication link establishment procedure. When the wireless interface 32 of the ultrasound machine 14 is brought into range of the wireless interface 20 of the patient monitor 12, the communication link establishment procedure could begin automatically, could be initiated through the user interface 28 of the patient monitor, and/or could be initiated through a user interface 42 of the ultrasound machine 14.

During the communication link establishment procedure, the patient monitor 12 and ultrasound machine 14 typically exchange information that identifies the type and capabilities of each. Further, the ultrasound machine 14 could send the patient monitor 12 data indicating the type, operating characteristics and/or modes operation of the transducer probe 38 connected to the transducer interface 40.

The communications processing unit 16 may be adapted/programmed to store information that identifies which ultrasound machines 12 are authorized to connect to the patient monitor 12. When the patient monitor 12 communicates with an authorized ultrasound machine 14, a private communication link could be established automatically such that the patient monitor 12 only communicates with the authorized ultrasound machine 14 and the ultrasound machine 14 only communicates with the patient monitor 12.

Alternatively, a user could be prompted through user interface 28 and/or user interface 42 to authorize the establishment of a private communication link between the patient monitor 12 and the ultrasound machine 14. Thus, in exemplary embodiments of the present disclosure, the controller unit 34 of the ultrasound machine 14 could activate an indicator 44 signaling that the communication link has been established. The indicator 44 could take the form of an audio and/or visual indicator.

Further, the communications processing unit 16 of the patient monitor 12 and/or the communications processing unit 30 of the ultrasound machine may be adapted/programmed to monitor the quality of the wireless communication link between the wireless interfaces 20, 32 and activate the indicator 44 if connectivity is lost or if data throughput is lower than a predetermined threshold. For example, if the latency of the communication link between the wireless interfaces 20, 32 exceeds 50 milliseconds, the indicator 44 may be activated or a message could be generated on the user interface 28 indicating that steps should be taken to improve the link quality. It is important that the latency of the communication link be as low as possible to ensure that the control data from the patient monitor 12 is received and processed by the ultrasound machine 14 for acquisition of ultrasound frames, images and/or volumes at an appropriate time during a cardiac cycle.

Although the present disclosure has been described with reference to exemplary embodiments and exemplary applications, the present disclosure is not limited thereby. Rather, the disclosed apparatus, systems and methods are subject to various changes, modifications, enhancements and/or alternative applications without departing from the spirit or scope of the present disclosure. Indeed, the present disclosure expressly encompasses all such changes, modifications, enhancements and alternative applications herein.

What is claimed is:

1. An ultrasound diagnostic imaging system (10), comprising:
a patient monitoring apparatus (12) including a communications processing unit (16) in electrical communication with a data processing unit (24), said communications processing unit (16) including a sensor interface (18) for receiving physiological data and a wireless interface (20) for transmitting ultrasound control data, said data processing unit (24) configured to generate ultrasound control data that includes timing data in response to said received physiological data; and
an ultrasound apparatus including a communications processing unit (30) in electrical communication with a controller unit (34) that is in electrical communication with a transducer probe (38), said communications processing unit (30) including a wireless interface (32) in wireless communication with said wireless interface (20) of said patient monitoring apparatus (12), said controller unit (34) configured to activate said transducer probe (38) to acquire ultrasound data during a physiological cycle based on said timing data of said ultrasound control data.

2. The ultrasound diagnostic imaging system (10) according to claim 1, wherein said physiological data includes electrocardiogram data.

3. The ultrasound diagnostic imaging system (10) according to claim 1, wherein said physiological data includes R-wave indicator data.

4. The ultrasound diagnostic imaging system (10) according to claim 1, wherein said physiological data includes respiration data.

5. The ultrasound diagnostic imaging system (10) according to claim 1, wherein said physiological data includes pulse data.

6. The ultrasound diagnostic imaging system (10) according to claim 1, wherein said physiological data includes oximetry data.

7. The ultrasound diagnostic imaging system (10) according to claim 1, wherein said data processing unit (24) is configured to generate ultrasound control data based on the type of said transducer probe (38).

8. The ultrasound diagnostic imaging system (10) according to claim 1, wherein said data processing unit (24) is configured to generate ultrasound control data based on the operational characteristics of said transducer probe (38).

9. The ultrasound diagnostic imaging system (10) according to claim 1, wherein said data processing unit (24) is configured to generate ultrasound control data based on the mode of operation of said transducer probe (38).

10. The ultrasound diagnostic imaging system (10) according to claim 1, wherein said communications processing unit (16) of said patient monitoring apparatus (12) and said communications processing unit (30) of said ultrasound apparatus (14) establish a private wireless communications link exclusively between said wireless interface (20) of said patient monitoring apparatus (12) and said wireless interface (32) of said ultrasound apparatus (14).

11. The ultrasound diagnostic imaging system (10) according to claim 1, wherein said communications processing unit (16) of said patient monitoring apparatus (12) and said communications processing unit (30) of said ultrasound apparatus (14) establish a wireless communications link having a latency of fifty milliseconds or less between said wireless interface (20) of said patient monitoring apparatus (12) and said wireless interface (32) of said ultrasound apparatus (14).

12. The ultrasound diagnostic imaging system (10) according to claim 1, wherein an indicator (44) is activated when said communications processing unit (16) of said patient monitoring apparatus (12) and said communications processing unit (30) of said ultrasound apparatus (14) establish a wireless communications link between said wireless interface (20) of said patient monitoring apparatus (12) and said wireless interface (32) of said ultrasound apparatus (14).

13. The ultrasound diagnostic imaging system (10) according to claim 1, wherein an indicator (44) is activated when a latency of a wireless communications link established between said wireless interface (20) of said patient monitoring apparatus (12) and said wireless interface (32) of said ultrasound apparatus (14) exceeds a predetermined threshold.

14. The ultrasound diagnostic imaging system (10) according to claim 1, wherein said data processing unit (24) generates control data indicating a particular ultrasound frame to be acquired during the physiological cycle.

15. The ultrasound diagnostic imaging system (10) according to claim 1, wherein said data processing unit (24) generates control data indicating a particular ultrasound image to be acquired during the physiological cycle.

16. The ultrasound diagnostic imaging system (10) according to claim 1, wherein said data processing unit (24) generates control data indicating a particular ultrasound acquisition volume to be acquired during the physiological cycle.

17. The ultrasound diagnostic imaging system (10) according to claim 1, wherein said ultrasound apparatus further includes a microbeamformer unit (36), said data processing unit (24) configured to generate ultrasound control data for said microbeamformer unit (36).

18. A method for communicating ultrasound data; comprising:
providing an ultrasound diagnostic imaging system (10) that includes a patient monitoring apparatus (12) having a communications processing unit (16) in electrical communication with a data processing unit (24), said communications processing unit (16) including a sensor interface (18) for receiving physiological data and a wireless interface (20) for transmitting ultrasound control data, said data processing unit (24) configured to generate ultrasound control data that includes timing data in response to said received physiological data;
providing an ultrasound apparatus including a communications processing unit (30) in electrical communication with a controller unit (34) that is in electrical communication with a transducer probe (38), said communications processing unit (30) including a wireless interface (32) in wireless communication with said wireless interface (20) of said patient monitoring apparatus (12), said controller unit (34) configured to activate said transducer probe (38) to acquire ultrasound data during a physiological cycle based on said timing data of said ultrasound control data;
effecting wireless communication between said ultrasound diagnostic imaging system (10) and said ultrasound apparatus.
